(19) United States
(12) Patent Application Publication
(10) Pub. No.: US 2023/0397995 A1
(43) Pub. Date: Dec. 14, 2023

(54) ELBOW JOINT PROSTHESES

(71) Applicant: HOWMEDICA OSTEONICS CORP., Mahwah, NJ (US)

(72) Inventors: Matthew Victor KARTHOLL, Fort Wayne, IN (US); Michel HASSLER, Saint Ismier (FR)

(73) Assignee: HOWMEDICA OSTEONICS CORP., Mahwah, NJ (US)

(21) Appl. No.: 18/354,710

(22) Filed: Jul. 19, 2023

Related U.S. Application Data
(63) Continuation-in-part of application No. PCT/US2022/070264, filed on Jan. 20, 2022.
(60) Provisional application No. 63/139,897, filed on Jan. 21, 2021.

(51) Int. Cl. A61F 2/38; A61L 27/18

(52) U.S. Cl. CPC A61F 2/3804; A61L 27/18; A61F 2002/30327

(57) ABSTRACT

A humeral implant configured for implantation at a distal end portion of a humerus. The humeral implant can be configured to articulate with a portion of an ulna. The humeral implant may include a body portion having a first end, a second end, and a side surface that extends between the first end and the second end, where the side surface includes a concave region that defines a first cross-sectional diameter that is a minimum cross-sectional diameter of the body portion.

### CROSS-REFERENCE TO RELATED APPLICATIONS

This application is a continuation-in-part of International Application No. PCT/US2022/070264, filed on Jan. 20, 2022, which claims priority to U.S. Provisional Application No. 63/139,897, filed on Jan. 21, 2021, the entire contents of which are incorporated herein by reference.

### FIELD OF DISCLOSURE

This application relates to an elbow joint prostheses and methods for assembling, fitting and implanting the same.

### BACKGROUND

Elbow prostheses are sometimes implanted in patients with deteriorating elbow joint function. The elbow joint function can deteriorate for a number of reasons, including osteoporosis, cartilage wear, trauma, and other reasons. The elbow is a complex joint involving three bones, with the joint being formed where the distal end of the humerus and the proximal ends of the radius and ulna meet. These bones are smaller than bones found at other more commonly replaced joints. As a result, the individual components are also smaller. Because the lower arm is highly mobile, artificial elbow joint components must be highly mobile and able to sustain a wide variety of loads without failing.

### SUMMARY

Some elbow prostheses can replace all three articular surfaces in the joint in a total elbow arthroplasty. Total elbow joint replacement components can suffer from problems including loosening, disengagement, wear, and fracture of the components. It can therefore be advantageous to only resurface an articular surface or replace a portion of the elbow joint. This approach may be called an elbow hemiarthroplasty. In an elbow hemiarthroplasty, the native articular surface of one or more of the distal humerus, proximal ulna and proximal radius is or are retained. As a result, the individual artificial components in an elbow hemiarthroplasty may articulate with the native bone. With fewer components implanted, the risk of loosening, disengagement, wear and fracture is lessened.

For the foregoing and other reasons, improved elbow joint prostheses and components therefore are desired.

Some aspects of the present disclosure are directed to a humeral implant. The humeral implant can include a body portion having a side surface having a contoured profile, such as a concave region. The concave region can be disposed between a first end and a second end of the body portion. The humeral implant can be configured for implantation at a distal end portion of a native humerus or a distal end portion of a humeral stem anchor. For example, the humeral implant can be configured to rotate about its longitudinal axis relative to the native humerus when implanted. In another example, the humeral implant can be configured to rotate about its longitudinal axis relative to the humeral stem anchor when implanted. The humeral implant can be configured to articulate with a portion of a native ulna or an ulnar implant. In some configurations, the body portion can be at least partially made of or coated with pyrocarbon (also known as pyrolytic carbon). In some embodiments, the body portion is made entirely of pyrocarbon to utilize the properties of pyrocarbon that can be beneficial for the implant's function and reliability.

Provided is a humeral implant configured for implantation at a distal end portion of a humerus. The humeral implant comprises a body portion that comprises: a first end and a second end; a side surface that extends between the first end and the second end, wherein the side surface comprises a concave region that defines a first cross-sectional diameter that is a minimum cross-sectional diameter of the body portion. The first end has a second cross-sectional diameter that is larger than the first cross-sectional diameter, and the second end has a third cross-sectional diameter that is larger than the first cross-sectional diameter.

A diameter of the humeral implant may vary along a length of the humeral implant. In this context, the length can include a direction from a first end surface to a second end surface. The direction corresponding to the length can be generally along the body portion, as viewed from the side. For example, the concave region can include a first diameter, and the first end can have a second diameter larger than the first diameter. The second end can have a third diameter larger than the first diameter and/or the second diameter. In some configurations, the first end and/or the second end may include a concave end surface. The first end may be at a first end of the humeral implant and the second end may be at a second end of the humeral implant. For example, in some configurations, the humeral implant may only consist of the body portion, e.g., without other components that are separable from prior to or after implantation. In some cases, the humeral implant consists of the body portion and surface coatings or features, as discussed below. For example, the humeral implant may be configured to only interface with the humerus and ulna, but not the radius.

Some aspects of the present disclosure are directed toward an elbow prosthesis. The elbow prosthesis can include a spool-shaped body configured to mimic, e.g., to replace, a trochlea portion of a distal humerus. The spool-shaped body can have a side surface that includes a concave region that forms the narrowest portion of the spool-shaped body. When implanted, the spool-shaped body can be configured to rotate about its longitudinal axis relative to the distal humerus. The concave region can be configured to receive a trochlea groove of a native ulna. The concave region can be configured to receive a groove of an ulnar implant. In some configurations, the spool-shaped body can comprise, e.g., be at least partially made of, or can be coated with pyrocarbon. In some embodiments, the spool-shaped body can be entirely made of pyrocarbon.

In some configurations, the first end of the spool-shaped body can include a first concave end surface and/or the second end of the spool-shaped body can include a second concave end surface. Each of the first end surface and the second end surface can be concave about a first axis. In some configurations, the first concave end surface can be concave in a first direction and the second concave end surface can be concave in a second direction opposite from the first direction. In some configurations, the concave region of the spool-shaped body can be concave about a second axis that is transverse to the first axis.

In some configurations, the first end of the spool-shaped body is at a first end of the elbow prosthesis and the second end of the spool-shaped body is at a second end of the elbow prosthesis such that the elbow prosthesis may consist of only the spool-shaped body. In some configurations, the spool-shaped body can have a length between about 15 mm and about 25 mm. The spool-shaped body can have a minimum diameter between about 10 mm and about 20 mm and/or a maximum diameter between about 15 mm and about 30 mm.

Some aspects of the present disclosure are directed towards a kit for treating an elbow joint. The kit can include a plurality of humeral implants of different sizes. For example, the plurality of humeral implants can include at least two or at least four different sizes.

Some aspects of the present disclosure are directed toward a method for treating an elbow joint. The method can include accessing an end portion of a humerus bone, sometimes referred to herein as a humerus. The method can also include, for example, forming a recess shaped to receive the humeral implant of the present disclosure at the distal end portion of the humerus. The method can include positioning the humeral implant at the distal end portion of the humerus bone. For example, the humeral implant can be positioned at the distal end portion of the humerus such that the humeral implant articulates against a portion of an ulna, e.g., against a native portion of the ulna or an ulnar implant.

In some implementations, an olecranon process of the ulna may at least partially constrains the humeral implant in place. Soft tissues of the elbow joint may also constrain the humeral implant in place.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. All illustrations in the drawing figures are schematic and are not drawn to scale. The following is a brief description of each of the drawings.

FIG. 1A illustrates a humeral implant implanted in an elbow joint.

FIG. 1B illustrates another view of the humeral implant implanted in the elbow joint.

FIG. 2A is a perspective view of a humeral implant.

FIG. 2B is a side view of the humeral implant of FIG. 2A.

FIG. 2C is another perspective view of the humeral implant of FIGS. 2A and 2B.

FIG. 3A illustrates an elbow in position for implanting a humeral implant in an elbow joint.

FIG. 3B illustrates exposure of the elbow joint for implanting the humeral implant in the elbow joint.

FIG. 3C illustrates removal of a portion of the humerus for implanting the humeral implant in the elbow joint.

FIG. 3D is an illustration showing a humeral implant of the present disclosure positioned in the prepared distal portion of the humerus shown in FIG. 3C.

FIG. 4 is an illustration showing another embodiment of the humeral implant of the present disclosure that is configured with a stem.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Disclosed are various embodiments of an elbow joint prostheses and methods that can be used in elbow joint replacement procedures, which can be used to correct elbow joint conditions including deformity, wear, osteoarthritis, and trauma. As discussed in greater detail below the apparatuses and methods herein reduce risk of disengagement, dislocation and decoupling, and also facilitate implantation and removal of the apparatuses during surgical procedures, and provide ranges of sizes to better fit a full range of patients.

### I. Elbow Joint Prostheses for Native Bone Interaction

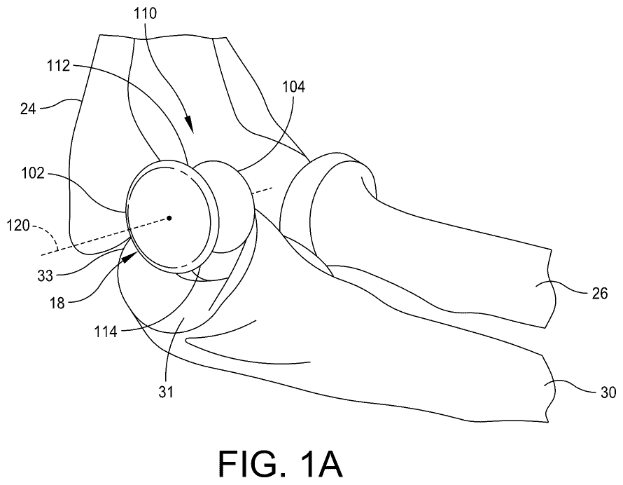

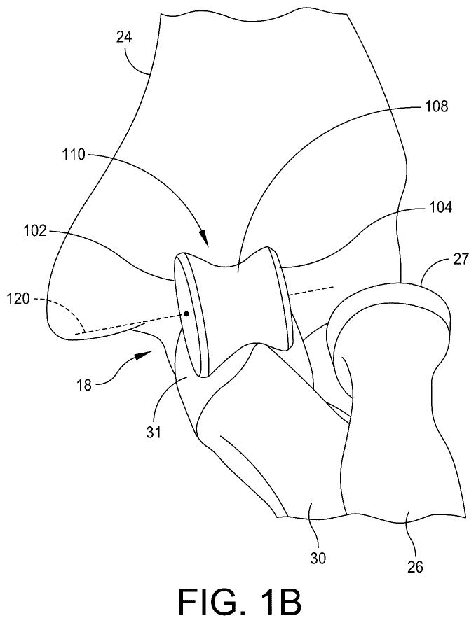

As shown in FIGS. 1A and 1B, the elbow joint can include several bone surfaces, including the distal humerus 24, the proximal radius 26, and the proximal ulna 30. The distal humerus 24 can include a trochlea, which can be configured to articulate with the ulna 30, and the capitellum, which can be configured to articulate with the radius 26. The proximal ulna 30 can include a trochlear notch 31 that is configured to receive the trochlea of the distal humerus 24. The proximal radius 24 can include a radial head 27 that is configured to articulate with the capitellum of the distal humerus 24.

Various embodiments disclosed herein relate to elbow prosthesis assemblies that can beneficially lead to improved patient outcomes, for example, by reducing the volume of bone removed from the patient's humerus, radius, and/or ulna, reducing surgery time, and improving reliability of the prosthesis.

In some conventional full elbow arthroplasty techniques, a humeral stem anchor may be inserted into the patient's distal humerus, a radial stem anchor may be inserted into the patient's proximal radius, and/or an ulnar stem anchor can be inserted into the patient's proximal ulna. The humeral stem anchor can be configured with an articular body to articulate with an articular body of the radial stem anchor and/or an articular body of the ulnar stem anchor. Such stemmed anchors may present long-term fixation issues, as well as undesirable radiologic signatures such as radiolucencies, spot welds, etc. Furthermore, traditional full arthroplasty techniques can fail for a number of reasons, such as dislodgement, early loosening, periprosthetic fractures, infection, instability, and wear. To reduce fixation problems, radiologic signatures, and surgery times associated with traditional stemmed anchors, stemless anchors can be used. Stemless elbow arthroplasty can result in shorter surgery time, less blood loss, and fewer periprosthetic fractures.

Hemiarthroplasty techniques can involve a partial elbow arthroplasty, where only a portion of the elbow joint is replaced. For example, elbow hemiarthroplasty can replace the distal humerus with an implant, but not the radial head or the proximal ulna. The hemiarthroplasty technique can offer several advantages, including retention of native bone, decreased dislodgement, decreased loosening, decreased wear, and decreased fracture of the implant.

Beneficially, various embodiments disclosed herein disclose a stemless implant that preserves native bone for use in subsequent procedures. Moreover, the stemless implants disclosed herein can be biocompatible to interact with native bone. A variety of elbow joint prosthesis assemblies and components are provided herein that include materials to provide enhanced compatibility for native bone interaction.

FIG. 1A shows a humeral implant 18 for a humerus-ulnar hemiarthroplasty, where the humeral implant 18 is implanted in the elbow joint at the distal end of the humerus 24 and is configured to articulate with the ulna 30. FIG. 1B shows another view of the humeral implant 18 implanted in the elbow joint. The humeral implant 18 is configured to interface with a portion of the native humerus 24 when implanted. The humeral implant 18 also can interface with a humeral stemmed anchor in some applications.

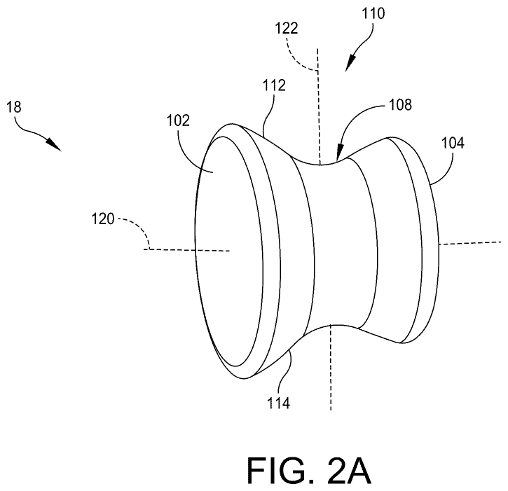

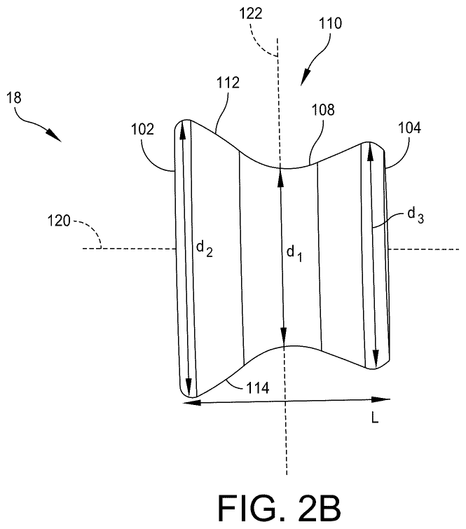

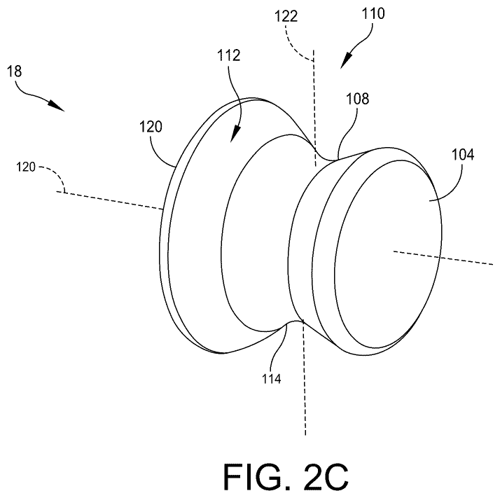

In some embodiments, the humeral implant 18 may be configured to rotate about its longitudinal axis 120 (see FIGS. 2A-2B) relative to the native humerus 24 after implantation. In some embodiments, the humeral implant 18 may be configured to rotate about its longitudinal axis 120 relative to a humeral stemmed anchor coupled with the native humerus 24 after implantation.

In some configurations, the humeral implant 18 can be a component of an elbow joint assembly configured to interact with other components of the elbow joint prosthesis, such as an articular body coupled with a humeral stemmed anchor. In other configurations, the humeral implant 18 can be the entire elbow joint prosthesis and interface directly with the native humerus 24 and the native ulna 30. The humeral implant 18 can provide smooth sliding motion over a distal surface of the humerus 24 and a proximal surface of the native ulna 30.

The humeral implant 18 is a stemless implant in the shape of a spool. In some embodiments, the spool shape can have a narrow waist, hereinafter referred to as a concave region. The humeral implant 18 can be shaped to match or approximate the anatomical shape of the trochlea of the humerus 24. The humeral implant 18 can be formed following pre-operative analysis of a particular patient, e.g., following obtaining a CT scan or other imaging of the elbow of a particular patient into which the implant 18 is to be implanted. The implant 18 can then be made to an appropriate size for the patient. In some modes, the humeral implant 18 can be supplied in a kit with a range of configurations for different groups of patients, e.g., different sizes. As shown in FIG. 1A, the humeral implant 18 can have a body portion 110 with a first end 102 and a second end 104. The second end 104 may be configured to face the radius when implanted. As shown in FIG. 1B, the body portion 110 can include a concave or necked region 108 extending between the first end 102 and the second end 104, e.g., from the first end 102 to the second end 104. The first end 102 may be at a first end of the humeral implant 18 and the second end 104 may be at a second end of the humeral implant 18 with the absence of structure beyond either end. The humeral implant 18 may consist of only the spool shaped body portion 110 in some embodiments. The humeral implant 18 can have a length, measured from the first end 102 to the second end 104, of at least about 15 mm and/or less than or equal to about 25 mm. The length may be measured along a straight axis that intersects the geometric center of the end 102 and the geometric center of the end 104.

The first end 102 and/or the second end 104 of the humeral implant 18 can be constrained by bone, which prevents displacement in the antero-posterior direction without limiting rotation of the humeral implant 18 relative to the humerus 24. Further, the humeral implant 18 can be positioned at the distal end portion of the humerus 24, such that an olecranon process 33 or trochlear notch 31 of the ulna 30 at least partially constrains the humeral implant 18 in place. Further, because the humeral implant 18 can be used in a hemiarthroplasty, the soft tissues may be retained, which would further limit displacement the humeral implant 18. Where multiple sizes of the humeral implant 18 are provided, the tension in the soft tissue may be one factor in considering the appropriate size of the implant.

As shown in FIGS. 2A-2C, the spool-like body portion 110 comprises a side surface 112 (like the side of a barrel) that extends between the first end 102 and the second end 104. The body portion 110 has a spool-like shape with a concave region 108 between the first end 102 and the second end 104. As can be seen in FIGS. 2A-2B, the side surface 112 can have a generally curved contour with a concavity that extends radially inward towards the longitudinal axis 120 of the humeral implant 18. This results in a shape where the concave region 108 includes a portion that is the narrowest part of the whole body portion 110 and, thus, has the smallest (i.e., the minimum) cross-sectional diameter d₁ of the body portion 110, as shown in the side view in FIG. 2B. Thus, the body portion 110 vary in diameter along the longitudinal axis 120 of the humeral implant 18 with the minimum cross-sectional diameter d₁ portion being in the concave region 108. The larger cross-sectional diameter near the first end 102 is d₂ and the larger cross-sectional diameter near the second end 104 is d₃.

As can be seen in the side view FIG. 2B, the transition zone 102′ from the first end 102 to the side surface 112 and the transition zone 104′ from the second end 104 to the side surface 112 are not abrupt but gradual and form curved surfaces. Therefore, the cross-sectional diameter d 2 is defined a small distance away from the first end 102, e.g., at a first local maximum diameter along the side surface 112 near the first end 102. The cross-sectional diameter d₃ is defined a small distance away from the second end 104, e.g., at a second local maximum diameter along the side surface 112 near the second end 104. In some configurations, a minimum diameter of the concave region 108 can be centered or positioned off-center and closer to one end of the humeral implant 18 than the other end of the humeral implant 18.

The contoured profile of the side surface 112 is configured to articulate or interact with a portion of a native ulna bone 30 or an ulna implant. The humeral implant 18 can be configured to interface with the native humerus 24 or a stemmed humeral anchor. In some embodiments, when implanted to the distal end of a humerus, the humeral implant 18 can be capable of rotating relative to the native humerus 24 or the stemmed humeral anchor. Alternatively, in some embodiments, when implanted to the distal end of a humerus, the humeral implant 18 can be affixed to the native humerus 24 such that it does not rotate.

The side surface 112 of the body portion 110 can have a contoured profile that is shaped to maintain contact with a portion of the ulna 30 or an ulna implant. For example, when viewed from side as in the view shown in FIG. 2B, the concave region 108 of the side surface 112 forms a pocket that keeps the portion of the ulna 30 in alignment with the humeral implant 18 to maintain contact. The concave region 108 can have a radius of curvature of at least about 10 mm and/or less than or equal to about 30 mm, such as between about 10 mm and about 20 mm, between about 15 mm and about 25 mm, or between about mm and about 30 mm. The radius of curvature may be constant or vary around the concave region 108. When the humeral implant 18 is designed to interface with a prosthetic implant, the side surface 112 can have a shallower curved profile (i.e., larger radius of curvature) where the concave region 108 defines the minimum cross-sectional diameter d₁ that is closer in value to d₂ and d₃, with a radius of curvature between about 20 mm and about mm. When the humeral implant 18 is designed to interface with a native bone, the side surface 112 can have a deeper curved profile (i.e., smaller radius of curvature) where the concave region 108 defines the minimum cross-sectional diameter d₁ that is further in value to d₂ and d₃, with a radius of curvature between about 10 mm and about 20 mm, for example between about 10 mm and about 15 mm. The contoured profile of the side surface 112 that includes the concave region 108 helps constrain the movement of the humeral implant 18 in a medio-lateral direction.

Generally, the cross-sectional diameter d₂ of the humeral implant 18 near the first end 102 and/or the cross-sectional diameter d₃ of the humeral implant 18 near the second end 104 can have a larger cross-sectional diameter relative to the concave region 108. For example, the humeral implant 18 can have a maximum cross-sectional diameter, measured near the first end 102 or the second end 104, between about 15 mm and about 30 mm. The humeral implant 18 can have a minimum cross-sectional diameter d₁, measured in the concave region 108, between about 10 mm and about 20 mm.

As illustrated in FIGS. 2A-2C, the humeral implant 18 can be asymmetrical. For example, the cross-sectional diameters d₂ and d₃ can be different. In some embodiments, the cross-sectional diameter d₂ near the first end 102 can be larger than the cross-sectional diameter d₃ near the second end 104. In some other embodiments, the cross-sectional diameter d₂ near the first end 102 is smaller than the cross-sectional diameter d₃ near the second end 104. In some other embodiments, the cross-sectional diameters d₂ and d₃ can be the same. The particular dimensions for d₁, d₂, and d₃ can be defined to better accommodate the particular bone dimensions of the patient's elbow joint.

According to another aspect of the present disclosure, the surface of the first end 102 can be concave about the longitudinal axis 120, meaning that the concave end surface is radially symmetric about the longitudinal axis 120. Similarly, the second end 104 can have a surface that is concave about the first axis 120. The concave surface of the first end 102 can be concave in a first direction, while the concave surface of the second end 104 can be concave in a second direction opposite from the first direction. In other configurations, the first end 102 and/or the second end 104 can be flat surfaces.

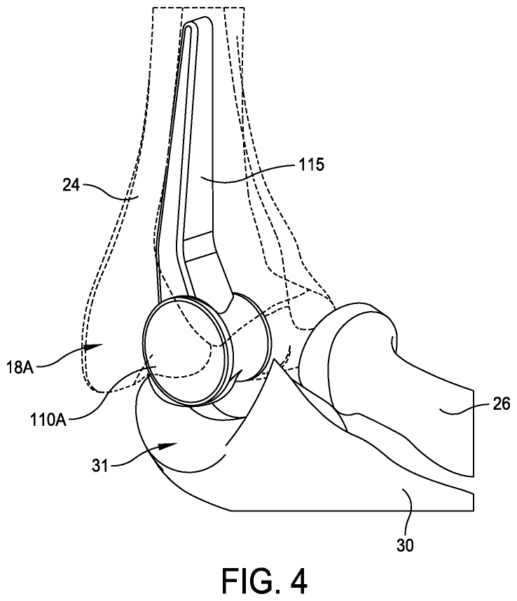

As illustrated, the humeral implant 18 may be stemless or unanchored to promote movement of the humeral implant 18 against the native humerus 24 or the native ulna 30 to stimulate fibrous cartilage growth. The humeral implant 18 may also be stemless for ease of manufacture. Alternatively, the humeral implant 18 can have one or more stems to fix the humeral implant 18 to the native bone and provide stability. FIG. 4 is an illustration showing an example of such humeral implant 18A configured with one stem 115. The stem 115 extends from the side surface 112 of the body portion 110A of the humeral implant 18A and is inserted into the humerus when the humeral implant 18A is installed into a patient.

In some embodiments, the humeral implant 18 is a solid unitary structure without any holes or recesses so that the implant 18 presents the smoothest possible surfaces to the surrounding tissues when implanted in an elbow. The humeral implant 18 can include a material suitable for articulation with native bone, such that the material has near natural wear characteristics, along with an elastic modulus that closely matches cortical bone. For example, the humeral implant 18 can include or be coated with a material, such as pyrocarbon, synthetic cartilage, or any other material with favorable tribological properties with native bone.

In some configurations, the humeral implant 18 can have a graphite core coated at least partially or entirely with pyrocarbon on an outer surface or on all outer surfaces. In some configurations, the humeral implant 18 can be coated with pyrocarbon. In some embodiments, the humeral implant 18 is made entirely of or coated entirely with pyrocarbon.

For example, pyrocarbon may stimulate tissue (e.g., cartilage) growth upon movement over a native bone surface. Alternatively or additionally, the humeral implant 18 can include various other materials, such as graphite, carbon fiber, titanium, stainless steel, plastic, other polymeric material, or other suitable biocompatible material.

A kit can include a plurality of, e.g., at least two or at least four, humeral implants 18 of various sizes. The various humeral implants 18 permit the implantation of a humeral implant 18 that matches the patient's anatomy. More or fewer than four sizes can be provided.

The humeral implants 18 for the elbow joint prostheses may be selected and implanted according to a range of one or more sizes for a given corresponding humerus or ulna. For example, a kit may include four different sizes of humeral implants 18, such as small, medium, large, or extra-large humeral implants 18. The different sizes of humeral implants 18 permit the humeral implant 18 to correspond with the respective humerus or ulna in the elbow joint. For example, as shown in FIG. 2B, each humeral implant 18 can include a first diameter d 1 in the concave region 108, a second diameter d₂ at the first end 102, and a third diameter d₃ at the second end 104. The humeral implant 18 can also have a length L measured between the first end 102 and the second end 104. Each incrementally larger size of the implant may include at least the same or greater length L and/or a larger first diameter d₁, second diameter d₂, and/or third diameter d₃. Each of the sizes can vary in dimension in length, first diameter, second diameter, and/or third diameter.

For example, as shown in the table below, a kit can be provided with four different sizes of humeral implants with the shown dimensions:

| | d₁ | d₂ | d₃ | L | |
|---|---|---|---|---|---|
| Small | 15 mm | 18 mm | 23.5 mm | 17 | mm |
| Medium | 16 mm | 20 mm | 25.5 mm | 18.5 | mm |
| Large | 18 mm | 22 mm | 27.5 mm | 20 | mm |
| Extra Large | 18 mm | 23 mm | 27.5 mm | 21.5 | mm |

### II. Implantation Methods for Elbow Joint Prostheses for Native Bone Interaction

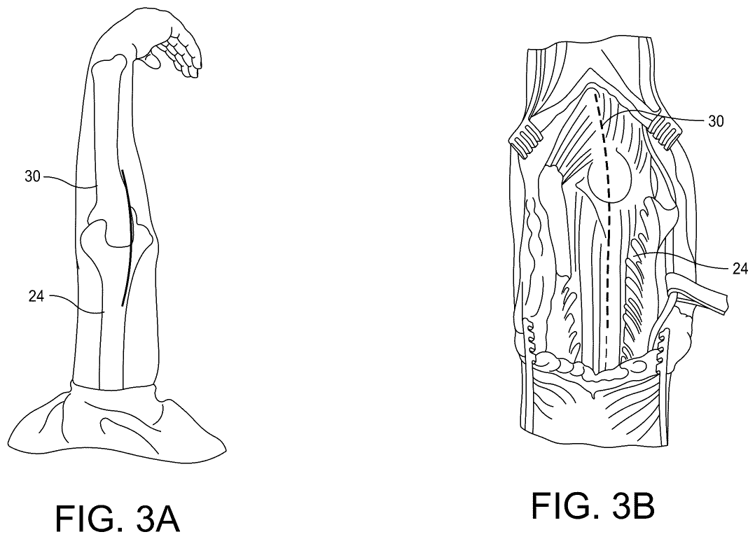

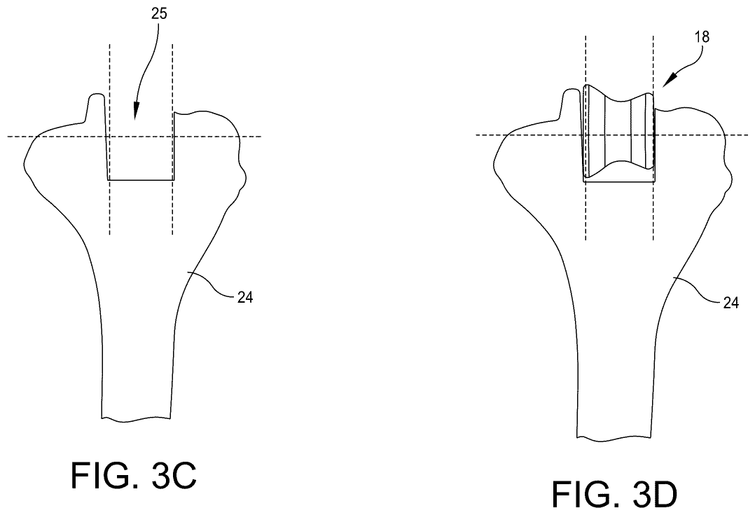

FIGS. 3A-3C show aspects of surgical methods related to the implantation of the humeral implants 18 as disclosed herein.

In preparation for the surgical procedure, the elbow can be in a lateral decubitus position or a supine position for implanting a humeral implant in an elbow joint (see FIG. 3A). After the elbow is in position, a straight incision can be created along the length of the arm to expose the elbow joint (see FIG. 3B). The tissue, such as the ligaments, muscle, and fascia, can be distracted or removed to expose or isolate the humerus 24 and the ulna 30. The bones of the elbow joint may also be isolated from the nerves so the nerves may be protected during the surgical procedure.

After the elbow joint has been isolated, the distal end portion of the humerus 24 can be accessed and prepared to receive a humeral implant 18. As shown in FIG. 3C, a portion of the distal end of the humerus 24 is removed with a tool, such as with a saw, to create a recess 25 in the distal end of the humerus 24 to receive the humeral implant 18. In some embodiments of the surgical procedure, the recess 25 has a contour that is a complementary negative of the contour of the side surface 112 of the humeral implant 18, in the distal end of the humerus 24. The humeral implant 18 can then be positioned in the recess 25, as shown in FIGS. 1A and 1B. In these methods, the elbow joint may be separated or dislocated during the procedure, but the position of the humeral implant 18 will be in the distal end of the humerus 24 and articulate against the proximal ulna 30 after the procedure.

Referring to FIG. 3D, once implanted, the humeral implant 18 is held in the recess 25 constrained by bone and tissue, but one side of the contoured profile of the side surface 112 is presented out of the recess 25 and toward the ulna 30. This configuration allows the side surface 112 of the humeral implant 18 to articulate with the ulna 30. In the embodiments where the humeral implant 18 is configured to rotate about its longitudinal axis 120, while positioned within the recess 25, the humeral implant 18 can rotate during its articulation against the ulna 30 as necessary. The humeral implant 18 can be positioned at the end of the humerus 24, such that an olecranon process of the ulna 30 at least partially constrains the humeral implant in place and/or soft tissues of the elbow joint at least partially constrains the humeral implant 18. When implanted, the humeral implant 18 may also be able to move relative to the humerus 24 as well. The motion of the humeral implant 18 relative to the humerus 24 can promote or stimulate growth of cartilage.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±1%, ±5%, ±10%, ±15%, etc.). For example, “about 15 mm” includes “15 mm.”

Although certain embodiments and examples have been described herein, it will be understood by those skilled in the art that many aspects of the delivery systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

1. A humeral implant configured for implantation at a distal end portion of a humerus, the humeral implant comprising:
a body portion that is made entirely of pyrocarbon and comprises:
a first end and a second end;
a side surface that extends between the first end and the second end, wherein the side surface comprises a concave region that defines a first cross-sectional diameter that is a minimum cross-sectional diameter of the body portion;
wherein the first end having a second cross-sectional diameter that is larger than the first cross-sectional diameter; and
wherein the second end having a third cross-sectional diameter that is larger than the first cross-sectional diameter.

2. The humeral implant of claim 1, wherein when implanted at the distal end portion of the humerus, the humeral implant presents a contoured profile of the side surface to an ulna or an ulna implant, wherein the contoured profile of the side surface articulates with a portion of the ulna.

3. The humeral implant of claim 1, wherein the humeral implant consists of the body portion.

4. The humeral implant of claim 1, wherein the second cross-sectional diameter is larger than the third cross-sectional diameter.

5. The humeral implant of claim 1, wherein the first end comprises a concave end surface.

6. The humeral implant of claim 1, wherein the second end comprises a concave end surface.

7. The humeral implant of claim 1, wherein the concave region comprises a radius of curvature between 10 mm and 30 mm.

8. The humeral implant of claim 1, wherein the humeral implant has a length between 15 mm and 25 mm.

9. The humeral implant of claim 1, wherein the humeral implant has a maximum diameter 15 mm and 30 mm.

10. The humeral implant of claim 1, wherein the body portion is at least partially made of pyrocarbon or comprises an outer surface of pyrocarbon.

11. The humeral implant of claim 1, wherein the humeral implant is configured to rotate about its longitudinal axis relative to the humerus when implanted.

12. A kit for treating an elbow joint, the kit comprising:
a plurality of humeral implants of claim 1 of different sizes.

13. The kit of claim 12, wherein the plurality of humeral implants comprises at least four different sizes.

14. An elbow prosthesis comprising:
a spool-shaped body extending along a longitudinal axis configured to be positioned at a trochlea portion of a distal humerus, the spool shaped body is made entirely of pyrocarbon and comprises:
a first end;
a second end; and
a concave surface extending between the first end and the second end, the concave surface configured to receive a trochlea groove of an ulna.

15. The elbow prosthesis of claim 14, wherein the elbow prosthesis consists of the spool shaped member.

16. The elbow prosthesis of claim 14, wherein the first end of the spool-shaped body comprises a first concave end surface that is concave about the longitudinal axis.

17. The elbow prosthesis of claim 16, wherein the second end of the spool-shaped body further comprises a second concave end surface that is concave about the longitudinal axis.

18. The elbow prosthesis of claim 17, wherein the first concave end surface is concave in a first direction, and wherein the second concave end surface is concave in a second direction opposite from the first direction.

19. The elbow prosthesis of claim 19, wherein the first end of the spool-shaped body is at a first end of the elbow prosthesis and the second end of the spool-shaped body is at a second end of the elbow prosthesis.

20. The elbow prosthesis of claim 15, wherein the concave surface comprises a radius of curvature between 10 mm and 30 mm.

21. The elbow prosthesis of claim 15, wherein the spool-shaped body comprises a length, measured along the longitudinal axis, between 15 mm and 25 mm.

22. The elbow prosthesis of claim 15, wherein the spool-shaped body comprises a maximum diameter between 15 mm and 30 mm.

23. The elbow prosthesis of claim 15, wherein the spool-shaped body comprises pyrocarbon.

24. The elbow prosthesis of claim 15, wherein the elbow prosthesis is configured to rotate about its longitudinal axis relative to the distal humerus when implanted.

25.-31. (canceled)
